(19) United States
(12) Plant Patent Application Publication
(10) Pub. No.: US 2012/0185978 P1
(43) Pub. Date: Jul. 19, 2012

(54) Pear tree named 'PREM 109'

(50) ;

(76) Inventor: Allan White, Havelock North (NZ)

(21) Appl. No.: 13/374,755

(22) Filed: Jan. 10, 2012

Related U.S. Application Data
(60) Provisional application No. 61/461,521, filed on Jan. 18, 2011.

(51) Int. Cl. A01H 5/00

(52) U.S. Cl. PLT/176

(57) ABSTRACT

A new and distinct pear tree named ‘PremP109’ is disclosed. The new pear is notable for its attractive appearance, superb texture, improved flavor and long shelf life.

### CROSS-REFERENCES TO RELATED APPLICATIONS

This Application claims priority to Provisional patent application No. 61/461,521, filed on 18 Jan. 2011, which is currently pending.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT-DISC

Not Applicable

### LATIN NAME OF THE GENUS AND SPECIES OF THE PLANT CLAIMED

Pyrus pyrifolia×Bretschneideri

### VARIETY DENOMINATION

‘PREMP109’

### BRIEF DESCRIPTION OF THE PHOTOGRAPHS

FIG. 1 shows the fruit of a “PremP109’ pear tree;

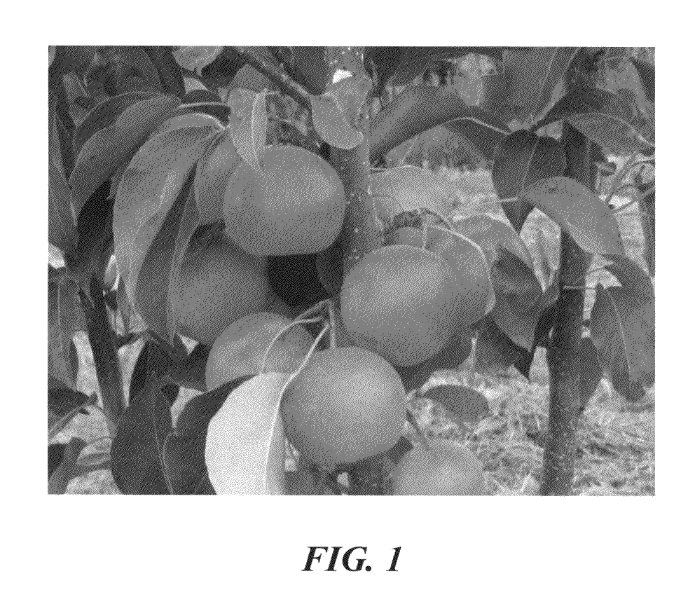

FIG. 2 shows a ‘PremP109’ pear tree;

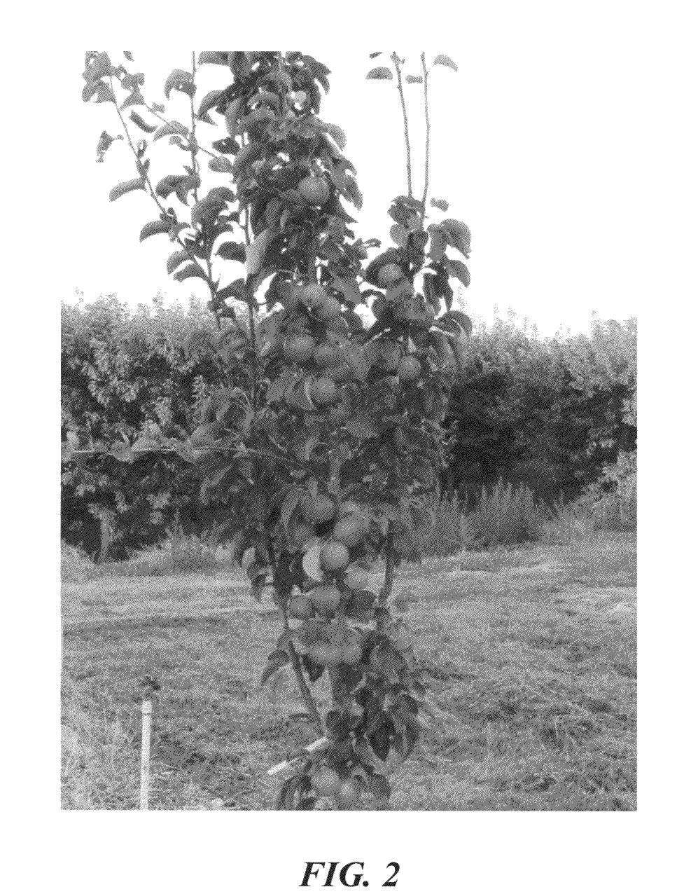

### BRIEF DESCRIPTION OF THE VARIETY

The new variety of pear tree ‘PremP109’ was created in the course of a planned breeding program carried out at The New Zealand Institute for Plant and Food Research, Hawkes Bay, New Zealand. ‘PremP109’ originated as a result of a controlled cross of ‘P125R74T032’ (see parent, not patented) and ‘P125R95T002’ (pollen parent, not patented). ‘PremP109’ was selected as a single plant from among a population of seedlings derived from the parents, and was selected for its attractive red color and unique appearance, superb texture and flavor, and long shelf life. Asexual propagation by budding was first carried out in Nelson, New Zealand. The variety has since been observed over a number of asexually propagated generations, and has been found to remain true to type.

### DETAILED BOTANICAL DESCRIPTION OF THE VARIETY

The following detailed botanical description is based on observations made during the 2010 growing season at Parker, Wash. All colors are described according to The Royal Horticultural Society Colour Chart. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant or any group of plants, of the new variety may vary from the stated average.
Tree: Vigor — Medium; Habit — Upright; Size — Height 213.4 cm, spread 45.7 cm; Trunk — Diameter at 5 cm above graft union 7 cm; Bark texture — smooth; Bark color — 166C.
Branch: Size — Diameter 2.5 cm at 1.37 m above graft union; Crotch angle relative to trunk — 45°; Branch color — over color 197D, under cover N167C; Lenticel density — 15 per square inch; Winter hardiness — Hardy; Chilling Requirement — Medium to high.
Dormant one-year-old shoot: Size — Diameter 1.5 cm, length 106.68 cm; color — 166B; Internode length — 4-5 cm; Lenticels — quantity 10 per square inch, color 155B, diameter 0.5 cm.
Flowers: Bud — Quantity per spur 8-9; Size — diameter 1 cm, length 1.5 cm, color — 65B, pink on edges; Petal — Quantity per flower 5; Touching, diameter 1.5 cm, length 2 cm, margin smooth; Sepals — Quantity per flower 5, diameter 0.2 cm, length 0.5 cm; Pedicel — Diameter 0.1 cm, length 2.5 cm, color 145A, Anthers — Quantity per flower 10, length 0.1 cm, pollen color 4C; Stigma — Size 0.1 cm, color 200D; Style — Length 0.4 cm, color 4C; Ovary — Size 0.3cm, color 151B; Bloom period — First bloom Apr. 5, full bloom Apr. 10 in Parker, Wash.
Leaf: Attitude in relation to shoot — upward; Blade margin — serrated; Size — length 12.2 cm, width 7.2 cm; Color — upper surface 146D; Color of lower surface — 145C; Apex with petiole — 2.8 cm; Attitude in relation to shoot — 45°.
Fruit: Size — diameter 8 cm, length 6.7 cm; General shape in profile round; Aperture of eye 21 cm; Size of eye 0.5 cm; Depth of eye basin 0.4 cm; Stalk — diameter 0.3 cm, length 4 cm, color 145C; Depth of stalk cavity 0.6 cm; Width of stalk cavity 0.8 cm; Size of lenticels 0.1 cm; Ground color of skin Yellow 12C; Over color of skin Red 33B; Flesh is crisp and very juicy; Flesh color 155B; Quantity of seeds 9; Seed color Black 202A.
Storage: Excellent storage performance of at least 10 weeks and up to 16 weeks and 14 days ambient shelf life.
Use: Fresh Market.

We claim:

1. A new and distinct pear tree substantially as shown and described herein.
